(19) United States
(12) Patent Application Publication
(10) Pub. No.: US 2017/0135751 A1
(43) Pub. Date: May 18, 2017

(54) ELECTROSURGICAL INSTRUMENT AND JAW PART FOR SAME

(71) Applicant: Aesculap AG, Tuttlingen (DE)

(72) Inventors: Christoph Rothweiler, Donaueschingen (DE); Eugen Herner, Villingen-Schwennningen (DE); Christian Huber, Mühlheim (DE)

(73) Assignee: Aesculap AG, Tuttlingen (DE)

(21) Appl. No.: 15/319,883

(22) PCT Filed: Jun. 15, 2015
(86) PCT No.: PCT/EP2015/063284

§ 371 Date: Dec. 19, 2016

(30) Foreign Application Priority Data
Jun. 25, 2014 (DE) 10 2014 108 914.6

(51) Int. Cl. A61B 18/14

(52) U.S. Cl. CPC A61B 18/1445; A61B 2018/00083

(57) ABSTRACT

An electrosurgical instrument includes a jaw part with instrument branches which are movable towards each other. One or more electrode surfaces are arranged on mutually facing sides of the branches. Relative movement of the instrument branches can be limited by at least one proximal spacer acting on proximal end portions of the instrument branches. At least one medial spacer acts on a medial portion, and at least one distal spacer acts on distal end portions of the instrument branches. The proximal and/or the distal spacer are made of electrically non-conductive material, and the at least one medial spacer includes at least one electrode made of an electrically conductive material and connected electrically conductively to the electrode. The at least one medial spacer interacts with a local insulation component made of a non-conductive material, which insulation component is arranged in an electrically insulating manner on at least one opposite electrode.

### RELATED APPLICATION(S)

This application is the United States national phase entry under 35 U.S.C. §371 of International Application No. PCT/EP2015/063284, filed Jun. 15, 2015, which is related to and claims the benefit of priority of German Application No. 10 2014 108 914.6, filed Jun. 25, 2014. The contents of International Application No. PCT/EP2015/063284 and German Application No. to 10 2014 108 914.6 are incorporated by reference herein in their entireties.

### FIELD

The present invention relates to an electrosurgical instrument, especially for laparoscopic operations, comprising a jaw part composed of instrument branches which are movable towards each other and on each mutually facing side of which one or more electrode surfaces are arranged/formed, wherein the movement of the instrument branches relative to each other is limited by a proximal spacer acting on proximal end portions of the instrument branches, a distal spacer acting on distal end portions of the instrument branches and at least one medial spacer acting on a medial portion of the instrument branches.

### BACKGROUND

After surgically resecting a hollow vessel section, e.g. during intestinal resection due to part of the intestines being affected by a tumor, the two hollow vessel cuts have to be reconnected at their opened ends so that a continuous course is formed. This is referred to as an end-to-end anastomosis. As a standard, the two opened ends are sewn together again, e.g. by clip suture instruments. Especially in the case of small and large intestine surgery, sometimes leaky suture connections (suture insufficiency) will occur involving a serious course of disease and also high mortality.

An alternative to sewing hollow vessel sections is the Tissue Fusion Technique (TFT) as it is called. TFT by means of high-frequency technology (HF) is based on denaturing proteins which are contained in a lot of tissues. In this way it is possible to weld tissue containing collagen. The tissue is heated during the welding operation to temperatures above the protein denaturing temperature and together with the intracellular and extracellular matrix is brought into a gel-like state. After compressing the tissue surfaces, the liquefied tissue is cooled to form a fused compound, thus causing a safe connection of the tissue.

For welding hollow vessel sections current flowing between electrodes at two clamping to jaws is applied to tissue seized between the two clamping jaws. In order to prevent failure of the sealing or welding, parameters acting on the tissue and being present during welding have to be detected and controlled. In order to guarantee this, an exact control of temperature, pressure, tissue impedance as well as distance and position of the clamping jaws is required.

In addition, it is desirable to treat tissue held between the clamping jaws uniformly so that all areas are reliably reached and there is no area to which excessive energy is applied. For this purpose, it has to be safeguarded that the HF electrodes are equally spaced apart from each other and, resp., are aligned in parallel to each other.

From the state of the art no instruments of an appropriate order of magnitude for use with the afore-mentioned hollow vessels and types of tissue are known. In the case of coagulation instruments of a rather small design, such as shown in EP 1 747 762 A2, by reason of the constructional design non-parallel alignment of the HF electrodes occurs upon closure of the clamping jaws, for example due to bending. This results in a reduction of the distance between the electrodes, and in the worst case short-circuits may occur.

On principle, the distance between the electrodes may be observed by spacers arranged at the clamping jaws. However, when a quite large number of spacers is provided on the clamping jaws, as is illustrated e.g. in EP 1 656 901 B1, EP 1 952 777 A1, EP 1 372 507 A1 or US 2004/122423 A1,the spacers necessarily perforate the tissue to be treated, as the tissue is compressed beneath the spacers when the clamping jaws are closed so that the tissue will be permanently damaged. This will affect the result of sealing.

When the pressure of the clamping jaws is reduced so as to avoid perforation of the tissue and the tissue is merely clamped beneath the spacers, an angular deflection of the clamping jaws will be resulting.

Since the spacers furthermore are made of electrically non-conductive material so as to avoid short-circuit between the HF electrodes, in the area of said spacers a so called coagulation shadow will form, i.e. the tissue sections are encapsulated in the area of or beneath the spacers, thus current is not or only insufficiently applied to said tissue sections and the vessel sections are not satisfactorily welded there. Moreover it has turned out that such electrically non-conductive spacers may easily chip off when they are fastened to the electrode for example by gluing, and then enter into a patient's body possibly without being noticed. In addition, the pre-defined electrode distance is no longer ensured in such case.

### SUMMARY

Against this background, the object underlying the present invention is to provide an instrument which by means of thermal fusion technique improves the result of an end-to-end anastomosis of hollow vessels, especially of hollow vessels such as small and large intestines, and generally in the field of tissue connections, in particular ensures parallel alignment of the HF electrodes without damaging the tissue and exhibits increased functional safety.

The object is achieved by an electrosurgical instrument comprising a jaw part composed of instrument branches which are movable towards each other and on each mutually facing side of which one or more electrode surfaces is/are arranged or formed, wherein the movement of the instrument branches relative to each other can be limited by a proximal (first) spacer acting on proximal end portions of the instrument branches, a distal (second) spacer acting on distal end portions of the instrument branches and at least one medial (third) spacer acting on a medial portion of the instrument branches, wherein the medial spacer is formed in that on an electrode a stop elevating from the electrode surface thereof is made of electrically conductive material and is connected electrically conductively to the electrode and interacts with an electrically insulating insulation component on the electrode surface of the opposite electrode, and the proximal spacer and/or the distal spacer is/are made of electrically non-conductive material.

A coagulation instrument for surgical purposes of the relevant species according to the invention includes, in one embodiment, instrument branches which are movable towards each other (preferably in a scissors or jaw-like manner) each having one or more electrode surfaces on the respective branch sides facing each other. Therebetween tissue may be clamped and electro-thermally treated. The movement of the instrument branches relative to each other is limited by at least one proximal spacer acting on proximal end portions of the instrument branches, at least one second distal spacer acting on distal end portions of the instrument branches and at least one medial spacer acting on medial portions. Proximal and distal spacers are made of electrically non-conductive material. Medial spacers have at least one projection made of electrically conductive material which keeps the opposed electrode surfaces at a distance from each other. Said projection is immediately provided and, resp., formed preferably integrally with the electrode by embossing or punching, for example, or is fixed on the same by welding or soldering, for example. On the opposite electrode an insulation component is inserted, preferably in the form of a pad or pin made of electrically non-conductive material, in an appropriate recess or is attached/glued thereon so as to form a contact surface for a conductive medial spacer so that the latter, although including conductive material, insulates the two opposite electrodes electrically against each other, however.

The embodiment according to the invention ensures that the distance of the opposed electrode surfaces is sized uniformly over the entire electrode surface. The combination of conductive medial spacers with non-conductive proximal and distal spacers allows taking advantage of the fact that due to the use of conductive spacers in the medial area of the electrodes uniform homogeneous current distribution, especially uniform current density, is brought about in the tissue clamped between the electrodes, while moreover non-conductive spacers are placed in the distal and proximal marginal areas of the electrodes where coagulation shadows are less relevant. Uniform surface pressure onto the tissue to be sealed can be achieved, thus causing an especially advantageous connection of the tissue. An adequately controllable and reproducible result of sealing of the said hollow vessels can be achieved. In this way leaks caused by non-conductive spacers in the central area of the electrode can be especially prevented from forming, wherein parallel alignment of the electrodes is constantly ensured. Moreover, by means of the non-conductive spacers in the proximal and distal marginal areas of the electrodes relatively large-surface electrically insulating bearing surfaces may be formed so that a uniform electrode distance may be adjusted and the risk of short-circuits with electrodes that are not fully covered may be reduced. It is another advantage that the size or area of the insulation component or insulation components of the medial spacers which, due to the instrument structure as a rocker arm, would have to be relatively large in order to avoid short circuits may be configured to be strongly reduced by using the non-conductive spacers at the proximal and, resp., distal electrode end so that the formation of coagulation shadows can be further reduced.

Summing up, the following advantages can be achieved, inter alia, by the invention: Safe tissue fusion by avoiding coagulation shadows, safe tissue fusion by continuously parallel alignment of the electrodes with each other, safe tissue fusion by a clearly defined distance of the electrodes, prevention of tissue damage by excessive force acting on the clamped tissue caused by the spacers, safe fusion of the individual tissue components by a constant balance of forces, avoidance of electric short circuits between the electrodes and avoidance of short circuits in the case of only partially covered electrodes.

According to the present invention, the medial spacers may be directly arranged and, resp., fixed on the electrode surfaces and may be formed of/connected to electrically conductive material integrally/soldered/welded with the respective electrode. On or at the opposite electrode for each medial spacer a non-conductive insulation component is provided or arranged on which the respective electrically conductive medial spacer is supported when the instrument is closed.

The electrically conductive medial spacer is preferably burl-shaped. The electrically insulating insulation components take for instance the shape of glued, applied, inserted or filled pads/platelets/pins substantially planar relative to the electrode surface, i.e. they have no or only a small projection from the respective electrode surface and are not or only hardly adapted to be detached/torn from the electrode surface. Accordingly, each of the insulation components can be dimensioned to be smaller with respect to the surface measures than an electrically non-conductive medial spacer according to the state of the art, as no shear forces have to be introduced to the electrode by the insulation component due to a small or missing projection. The medial spacers themselves may be made of a material such as metal that withstands high shear forces so that they can likewise be dimensioned to be small. In total this contributes to avoiding coagulation shadows and at the same time to increasing the functional safety.

It is of advantage when the insulation components are inserted in the form of pads or pins into appropriate indentations or recesses (troughs) on the surface of the respective electrode so that they form a substantially planar surface with the electrode (without any projection). It is also advantageous when each insulation component takes the shape of a pin having a flat plate portion to the lower side of which a pin extension is attached. Said pin extension is inserted in a corresponding bore inside the electrode and thus causes an even tighter seat/support of the insulation component in the electrode recess.

In addition to the afore-described measures, it may be provided to minimize the number of medial spacers fixed onto the respective electrode surface and hence of non-conductive insulation components used so as to additionally reduce the effects of coagulation shadows.

By providing the non-conductive proximal and distal spacers the electrodes are basically ensured to have a predetermined distance from each other over their entire length and thus to extend preferably in parallel to each other. By reason of the large distance of the spacers and, resp., their points of action in the longitudinal branch direction, the parallelism of the instrument branches and the electrode surfaces arranged thereon is improved, as when forming the spacers possible manufacturing tolerances thus have only little impact on the parallelism of the branches in the closing position. The substantially uniform electrode distance between the two branches adjustable in this way in the closing position provides for uniform penetration of the tissue with HF energy and for uniform current density within the tissue.

Damage of tissue and nonhomogeneous penetration of the same with HF energy can be further minimized in an embodiment of the invention by the fact that an as small number of medial spacers as possible are applied to each electrode surface. Preferably the instrument includes two or three medial spacers, especially preferred exactly one medial spacer.

The coagulation clamp according to the invention and, resp., the instrument branches according to the invention of such coagulation clamp thus create an optimum compromise between maximum parallel alignment of the HF electrodes in the closing position of the branches, on the one hand, and homogenous tissue fusion with minimum damage of tissue, on the other hand. Therefore, tissue damage caused by the spacers due to excessive force acting on the clamped tissue is prevented and safe fusion of the individual tissue components is ensured by constant force ratios, by the parallel arrangement of the electrodes, by the clearly defined distance of the electrodes and by the homogenous current distribution within the tissue along the electrodes. Moreover, by the specific arrangement of the non-conductive proximal and distal spacers which may be arranged or formed especially outside the electrode surfaces short-circuits between the electrodes and leaks on the sealed tissue layers are prevented. In this way, constant prerequisites are set for HF surgery especially with respect to the tissue impedance so that the quality of the sealed tissue areas can be better electrically controlled.

In accordance with another or additional aspect of the invention, the spacers made of electrically non-conductive material are arranged exclusively outside an area provided for treating the tissue at the proximal and distal ends of the branches. When non-conductive spacers act merely on the proximal and distal end portions of the instrument branches and the medial area of the instrument branches usually constituting the actual or substantial treatment area of the tissue is free of non-conductive spacers, coagulation shadows usually caused by the latter are avoided in this main area.

According to a further or different aspect of the present invention, at least one spacer, especially the proximal spacer, is realized in the form of a spacer module configured separately from the instrument branches. Said module may include at least one electrically non-conductive material tongue which in a closing position of the instrument branches is clamped between the latter. The height of the material tongue therefore corresponds to a predetermined (parallel) distance to be adjusted between the instrument branches.

A separate spacer module of this design offers plural advantages. On the one hand, it is easy to manufacture independently of the respective instrument branches and, resp., the coagulation clamp for which it is intended to be used. On the other hand, it can be exchanged at any time, either for reasons of wear or for replacement with a different spacer module having higher or lower material tongues. In this way the distance of the instrument branches in the closing position may be varied. The physical separation of instrument branches and spacers thus offers the advantage that the same spacer module can be provided for different instrument branches or that for the same instrument branches different spacer modules can be provided. Also, the coagulation shadow effect turns out to be smaller in the case of a material tongue which is loosely held as well as clamped between the electrode surfaces in the closing position of the branches than in the case of a spacer fixed on the electrode surfaces, even when the spacer consists of electrically conductive material and interacts with an insulation component made of electrically non-conductive material.

The spacer module may include plural material tongues (made of non-conductive material) spaced from each other laterally or in the transverse direction of the branches in order to spare e.g. an electrical cutting portion provided between two coagulation electrode surfaces.

For opening and closing the instrument branches at least one of the instrument branches may be pivoted e.g. on an instrument shaft or on the opposite branch and may be operable via a handling mechanism (supported in the instrument shaft and/or in the handle piece) so as to move the instrument branches towards and away from each other. The spacer module may be rotatably supported in a pivot joint of the operable (mounted) instrument branch, especially encompassed in a housing-like manner by joint portions of the operable instrument branch.

By integrating the spacer module into the pivot joint of one or both instrument branches said spacer module is not only accommodated in a space-saving manner inside the instrument or jaw part, but it exerts its spacer function independently without any additional actuation by the surgeon when the instrument branches are closed.

Alternatively or additionally to the afore-mentioned separate spacer module, at least either of the instrument branches, preferably the pivoting branch, may include a rotation limiting pin guided in a connecting link on the side of the other branch, the interaction of the rotation limiting pin and the connecting link simulating or constituting a kind of spacer, especially the spacer acting on the proximal end portions of the instrument branches, and the instrument branches adopting a predetermined distance from each other, when the at least one rotation limiting pin reaches an end portion of the connecting link. In case that both instrument branches are pivotally or otherwise movably (e.g. displaceably) mounted, the degree of freedom of each of the two instrument branches can be limited by a rotation limiting pin guided in a respective connecting link.

This solution especially offers the advantage that the spacer can be arranged at least in the proximal end portion of the branches completely outside the clamping area of the instrument branches, i.e. outside the tissue treatment area (electrode surfaces), and that there will be no contact between the spacer and the tissue to be treated. In this way any damage of the tissue can be safely prevented.

A spacer, especially the distal spacer, may be formed by a (burl-shaped) projection arranged outside the electrode surfaces, especially between two electrode surfaces, and facing the other instrument branch. Hence, when said spacer is not arranged on the electrode surfaces but next to them or therebetween, in this area no coagulation shadows will form, in particular also because in such case the spacer(s) need not be made of insulating material. When the spacer is arranged between the electrode surfaces, especially when it is arranged on the central axis of either of the instrument branches without being in direct contact with the electrode surfaces, no electric short-circuit will occur between the electrode surfaces. Moreover, there will be no torsional wear of the instrument branches when they are pressed against each other in the closing position and are kept apart merely by the spacer. In this way, the total number of spacers can be further reduced.

One or more spacers can be formed by only one projection directly provided/fixed on one electrode surface or by plural projections directly provided/fixed on respective different electrode surfaces. Thus, on the one hand, parallel alignment of the electrodes in the longitudinal direction is ensured and, on the other hand, the number of the spacers fixed on the electrode surfaces is kept small, thus enabling optimum treatment, especially homogeneous fusion of the tissue.

Between the proximal, medial and distal spacers at least on one instrument branch additionally one or more (burl-shaped) elevations facing the other instrument branch may be formed having a height which is smaller than the height of the spacers and especially amounts to 10% to 75% of the height of the spacers. Said elevations or teeth allow the tissue to be better held so as to prevent the tissue to be treated or the tissue portions to be treated from slipping out of the instrument branches before they have been fused to each other. Since said elevation is lower than the minimum distance of the two instrument branches defined by the spacers in the closing position, e.g. 10% to 75% of the distance, it will never get into contact with the opposite instrument branch. Therefore the tissue is not perforated between the elevation and the opposite instrument branch and hence is not permanently damaged. Furthermore, no coagulation shadows will form by said elevations, either, as the tissue clamped between the elevation and the opposite instrument branch is not substantially covered. The coagulation shadows formed by the conventional instruments are thus prevented. Since said elevations do not contact the opposite instrument branch anyway, they can equally be made of electrically conductive material, wherein in this case opposite pads/pins made of electrically non-conductive material can be dispensed with. As a matter of course, for each electrode surface plural elevations of this kind may be arranged. They may be arranged at regular intervals.

It has to be noted that both individual ones and plural of the afore-stated aspects and features may be combined with each other.

### BRIEF DESCRIPTION OF THE DRAWING FIGURES

FIG. 1 illustrates an electrosurgical instrument according to a first embodiment of the invention;

FIG. 2 illustrates an electrosurgical instrument according to a second embodiment of the invention;

FIG. 3 shows a perspective view of two instrument branches of the electrosurgical instrument pivoting (scissors-like) relative to each other including an enlarged view of a spacer module;

FIG. 4 shows a lateral view of the two instrument branches of FIG. 3 in an opened position;

FIG. 5 shows a lateral view of the two instrument branches of FIG. 3 in a closed position;

FIG. 6A illustrates detailed view A of FIG. 4;

FIG. 6B illustrates detailed view B of FIG. 5;

FIG. 6C illustrates detailed view C of FIG. 5;

FIG. 7 shows a perspective view of two instrument branches of an electrosurgical instrument pivotal relative to each other according to the invention;

FIG. 8 shows a lateral view of the two instrument branches of FIG. 7 in a closed position;

FIG. 9A shows detailed view A of FIG. 8;

FIG. 9B shows detailed view B of FIG. 8;

FIG. 9C shows detailed view C of FIG. 8;

FIGS. 10, 11 and 12 illustrate medial spacers in various detailed views;

FIG. 13 shows an electrosurgical instrument according to a further embodiment of the invention;

FIG. 14 shows the detailed view of a spacer as well as of an insulation component according to a first embodiment of the invention;

FIG. 15 shows the detailed view of a spacer as well as of an insulation component according to a second embodiment of the invention; and

FIG. 16 shows the detailed view of a spacer as well as of an insulation component according to a third embodiment of the invention.

### DETAILED DESCRIPTION

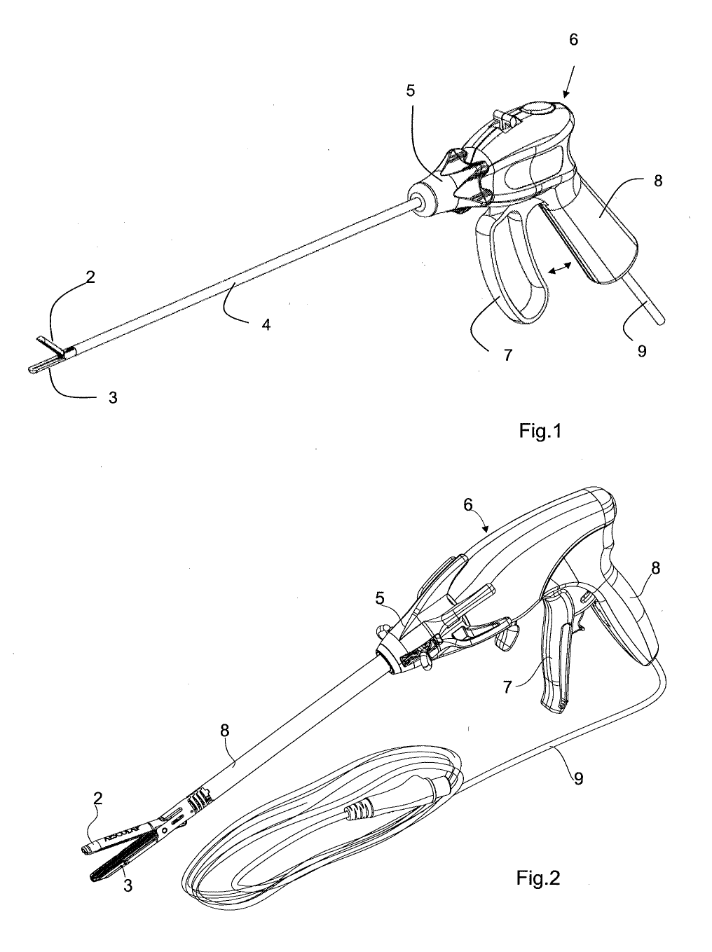

FIG. 1 illustrates a perspective view of a laparoscopic electrosurgical instrument 1 according to a first embodiment of the invention comprising a jaw part consisting of a pair of instrument branches 2 and 3 preferably movable towards each other in a scissors-like or forceps-like manner in an opened position which are arranged at the distal end of an instrument shaft 4 which in turn is rotatably fastened to a handle piece or handle part 6 via a manually operable shaft rotation means 5. Via the shaft rotation means 5 the shaft 4 and the instrument branches 2 and 3 disposed thereon can be rotated about the longitudinal shaft axis relative to the handle part 6. The handle part 6 includes a manually operable handle or trigger 7 which is pivotally movable relative to a hand or pistol grip 8 tightly connected to the handle part 6. The instrument branches 2, 3 or at least one manually operable instrument branch 3 is/are in operative connection with the handle 8 via an actuating mechanism (not shown in detail) such as a control cable or a push rod inside the instrument shaft 4 and can be brought preferably infinitely from an opened position into a closed position (and vice versa) by manually operating the handle 8. Via a line (shown in part only) or an electric cable 9 the handle part 6 is connected to an HF energy source (not shown) which allows applying HF voltage between the instrument branches 2 and 3 for an electrothermal treatment of tissue.

As regards the basic functioning and the mechanical structure of the instrument 1, especially the actuating mechanism, the published document WO 2011/097469 A2 is referred to.

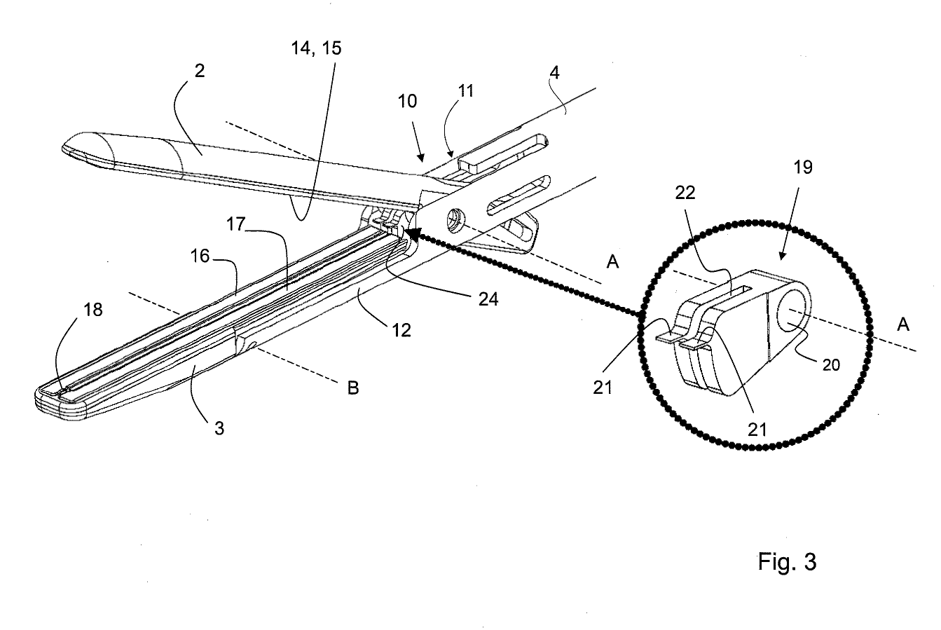

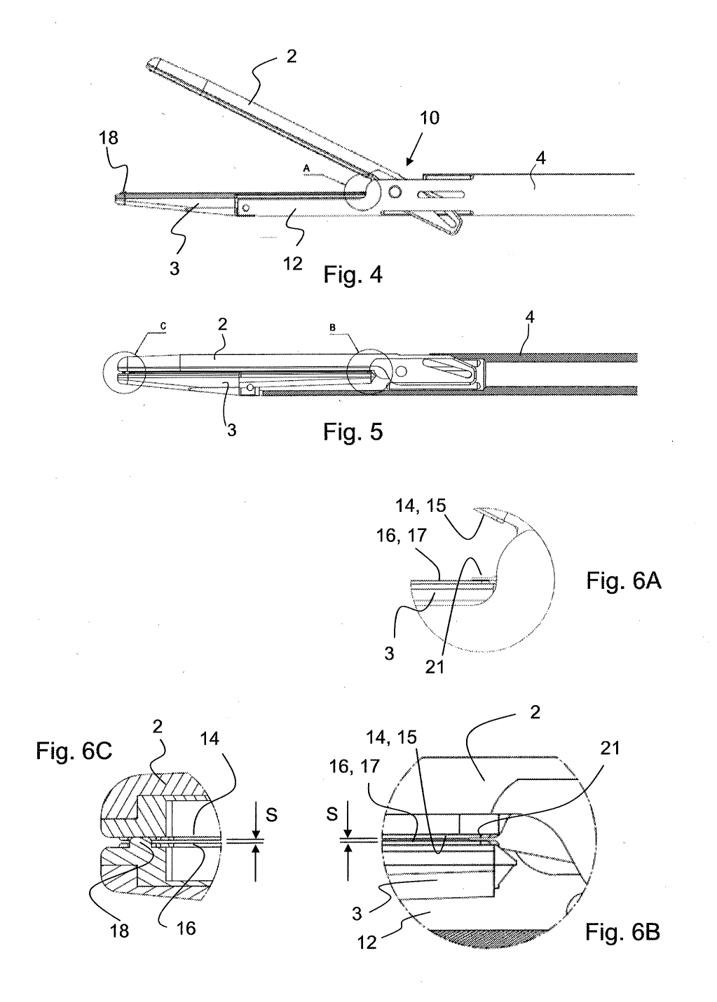

FIGS. 3 and 4 show in detail the distal end of the shaft 4 and, resp., the jaw part connected to the shaft 4 including the instrument branches 2 and 3 in an opened position. The first upper instrument branch 2 according to FIG. 3 or 4 is pivoted by a proximal pivot joint or hinge 10 (cf. FIG. 4) about a transverse axis A on the distal end of the shaft 4. For this, the distal shaft end or the jaw part connected thereto includes a centrally configured through slit or longitudinal gap 11 extending along the shaft each side wall of which has a (coaxially orientated) cross-bore defining the afore-mentioned transverse axis A. The through slit 11 further has a slit width that allows movably/pivotally inserting the first instrument branch 2 into the same. In an axial distal extension (projection) of the through slit 11 the distal shaft end or jaw part forms a supporting protrusion or support 12 in the form of a half-shell or groove which faces the rotatable upper instrument branch and on its distal end portion includes a through cross-bore substantially in parallel to the transverse axis A.

The second lower instrument branch 3 according to FIG. 3 or 4 is (axially) accommodated, viewed in the longitudinal shaft direction, only over a partial length portion in the shell-type supporting protrusion 12 so that it projects from the remaining partial length portion thereof axially from the supporting protrusion 12 in the distal direction. Further, the lower instrument branch 3 is pivotally hinged centrally in a rocker-like manner to the supporting protrusion 12 via the distal through cross-bore. By means of a spring mechanism (not shown) (cf. inter alia also WO 2011/097469 A2) the front or distal rocker part of the lower instrument branch 3 according to FIG. 4 is biased upwards and, resp., toward the upper instrument branch 2, thus causing the lower branch 3 to adopt, viewed in the longitudinal direction, a small angle with the instrument shaft 4 and with the supporting protrusion 12 and thus, when the jaw part closes, the distal end portions of the two branches 2, 3 first to get into clamping contact in a forceps-like manner. This facilitates gripping of tissue. The lower instrument branch 3 is held at the portion or supporting protrusion 12 in a rocker-type manner pivoting about the axis B defined by the through cross-bore only so far that minor angular deviations between the upper and lower instrument branches 2 and 3 can be compensated in the closing position and, resp., parallel alignment of the two branches 2, 3 can be achieved.

Each of the instrument branches 2 and 3 preferably includes, according to the present embodiment, two electrodes or electrode surfaces 14, 15, 16, 17 spaced in the transverse branch direction and extending substantially in parallel in the longitudinal branch direction to which HF voltage can be applied. Accordingly, when tissue is provided between the instrument branches 2 and 3 in their closed position, the surgeon is able to coagulate, separate or weld said tissue by the electrode surfaces 14, 15, 16, 17. Moreover, a specific electrosurgical knife (not shown) or an appropriate cutting means which is electrically insulated against the electrode surfaces 14, 15, 16, 17 may be arranged between the electrode surfaces 14, 15, 16, 17.

In order to avoid short-circuit between the electrode surfaces 14, 15, 16, 17 of the two instrument branches 2 and 3 and, respectively, to safeguard that homogeneous current flows across the tissue clamped between the electrode surfaces 14, 15, 16, 17 along the entire electrode length, the electrode surfaces 14, 15, 16, 17 have to remain substantially evenly spaced apart from each other also in the closed position. The instrument 1 therefore includes at the distal end portion of the lower instrument branch 3 (and/or the upper instrument branch 2) between the two electrode surfaces 16 and 17 a preferably burl-shaped projection 18 protruding from the electrode surfaces 16 and 17 by a predetermined degree corresponding to the desired distance between the electrode surfaces 14, 15, 16, 17, said projection 18 contacting the upper instrument branch 2 (and/or the lower instrument branch 3) upon closure of the jaw part and thus serving as a spacer at the distal end portions of the two instrument branches 2 and 3. According to this embodiment, at the proximal end portions of the two instrument branches 2 and 3 the distance between the electrode surfaces 14, 15, 16, 17 is brought about by a separate spacer module 19 which is freely held separated from the branches 2, 3 and, resp., from the electrodes 14, 15, 16, 17. Said spacer module 19 in the present case is a cam-shaped component having a proximal bearing portion (cam portion including through cross-bore) which is adapted to be engaged in the pivot joint (pivot bolt) 10 and which thus may rotate freely about the pivot axis A between the instrument branches 2 and 3. For reasons of space, in the present case the movable upper (and/or lower) branch 2 is hollowed at its proximal end portion in the area of the pivot axis A in the longitudinal direction, thus resulting in a kind of receiving space or longitudinal groove whose dimensions are sufficient for receiving the spacer module 19 therein. I.e. at least in the closing position of the jaw part the spacer module 19 is received between two groove walls of at least the one operable instrument branch 2.

FIG. 3 illustrates an enlarged perspective view of the spacer module 10 alone. As has been indicated in the foregoing already, the module 19 takes a kind of cam shape with the proximal bearing portion in which the cam has such cam thickness/height that it can be movably immersed in the proximal longitudinal groove of the one branch 2. In the bearing portion of the module 19 further the through cross-bore 20 is formed. On the distal transverse outside of the module 19 opposed to the bearing portion two flat material tongues 21 projecting radially with respect to the pivot axis A are integrally formed with their respective flat sides facing the branches 2, 3 and their tongue thickness/height H corresponding to a minimum distance S (clearance) to be obtained between the opposite electrode surfaces 14 and 16 and, resp., 15 and 17 in the closing position of the branches 2, 3 and their lateral distance (in the transverse branch direction) and width substantially corresponding to the parallel distance and the width of the electrode surfaces 14, 15, 16, 17 so that the material tongues 21 come to rest at least partially on the electrode surfaces 14, 15, 16, 17. Between the two material tongues 21 in the cam-shaped module 19 a longitudinal slit 22 open at the distal end of the module 19 is formed which extends up to the bearing portion and ends directly ahead of the cross-bore 20. The entire spacer module 19 or at least the material tongues 21 are made of electrically non-conductive material in this embodiment.

FIGS. 4 and 5 illustrate a side view of the jaw part and, resp., of the instrument branches 2 and 3 in the opened position and in the closed position. FIGS. 6A, 6B and 6C show detailed views of the jaw part according to FIGS. 4 and 5.

FIG. 6A illustrates that the material tongues 21 of the spacer module 19 loosely rest on a proximal end portion of the electrode surfaces 16 and 17 of the lower instrument branch 3, when the jaw part is in the open position. When the instrument branches 2 and 3 are brought into the closed position by preferably actuating the upper instrument branch 2 (cf. FIG. 5), the material tongues 21 of the separate spacer module 19 are clamped between the proximal end portions of the electrode surfaces 14, 15, 16, 17 of the two instrument branches 2 and 3 (cf. FIG. 6B) and the projection 18 at the distal end portion of the lower instrument branch 3 gets into contact with the distal end portion of the upper instrument branch 2. Hence the proximal and distal end portions and, consequently, also the entire electrode surfaces 14, 15, 16, 17 remain spaced from each other by the predetermined clearance S and substantially in parallel to each other.

As stated in the foregoing, the (burl-shaped) projection 18 in this embodiment is arranged between the electrodes and thus gets into direct contact with the upper operable branch 2 (rather than with the upper electrode surfaces of the branch 2). Moreover, the proximal material tongues 21 are not fixed directly to the electrode surfaces but are only adjacent thereto. Thus, in the first embodiment no spacer is provided directly on one of the electrode surfaces 14, 15, 16, 17 (i.e. fixed thereon). Hence coagulation shadow effects can be reduced as compared to the state of the art.

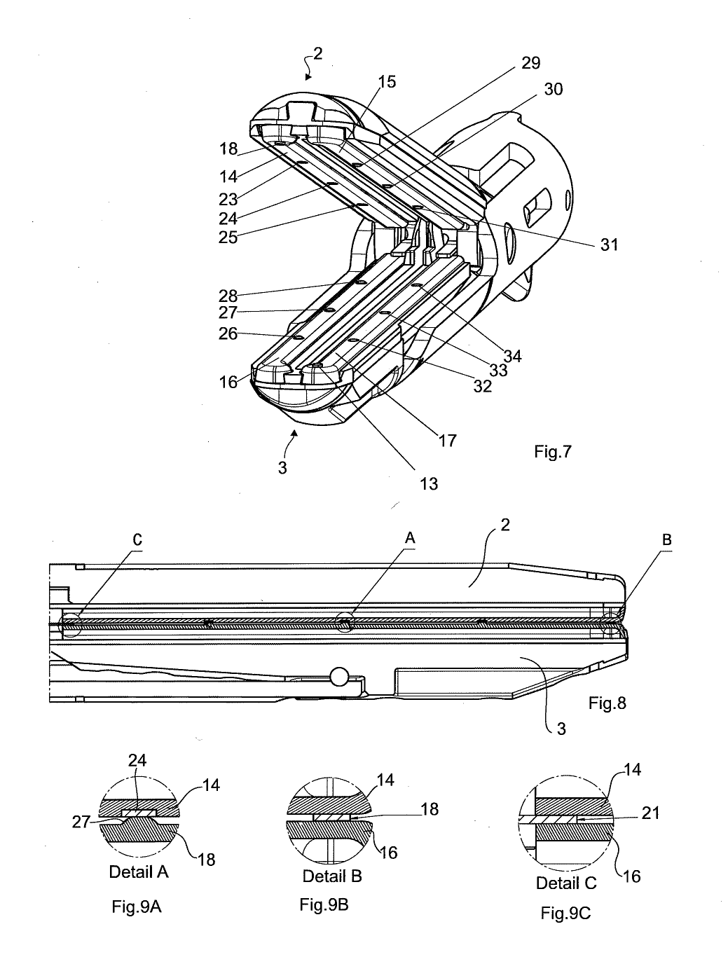

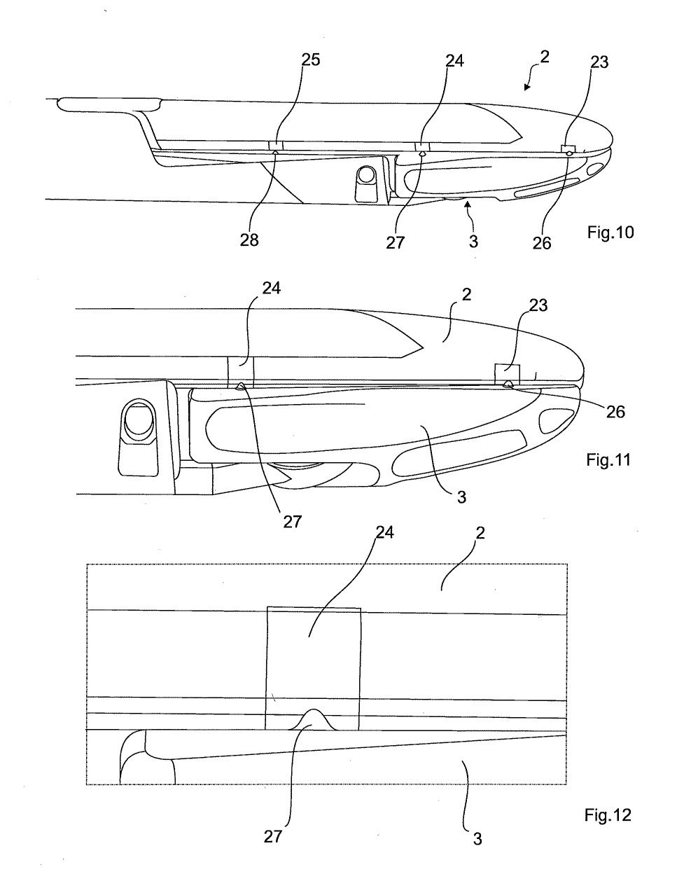

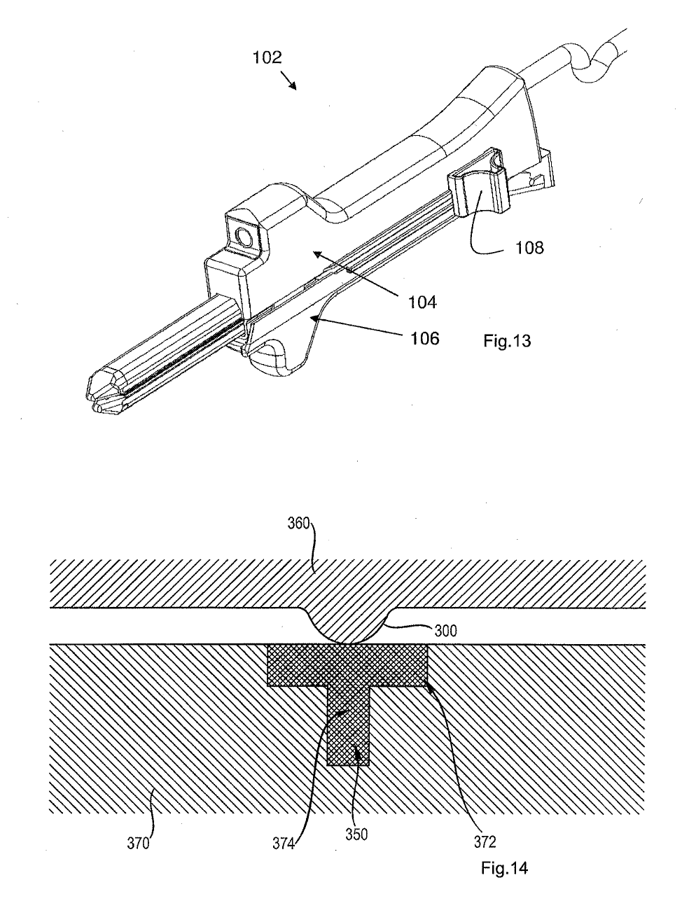

FIG. 7 shows an embodiment of the laparoscopic electrosurgical instrument 1 in a perspective representation. FIG. 8 shows the distal end portion of the instrument of FIG. 7 in a lateral view. FIGS. 9A, 9B and 9C illustrate enlarged cutouts each of which is marked in FIG. 8. FIG. 9A shows a medial spacer, FIG. 9B shows a distal spacer and FIG. 9C shows a proximal spacer. On the electrode surface 14 of the instrument 1 a projection 18 is arranged at the distal end and on the electrode surface 17 a projection 13 is arranged at the distal end, especially directly (i.e. tightly fixed) on the distal end portions of the electrode surfaces 14 and 17, respectively. The projections 13 and 18 form non-conductive distal spacers in accordance with the invention. In order to be able to avoid short-circuit between the electrode surfaces 14, 15, on the one hand, and between the electrode surfaces 16, 17, on the other hand, when they adopt a closed position close to the respective opposed electrode surfaces, the projections 13, 18 are made of an electrically non-conductive material. Therefore, they can abut against and thus contact the respective opposite electrode surface either directly or else indirectly, for example via insulation components not shown in FIG. 7 which may be provided (in point/pad shape) especially on the respective opposite electrode (exclusively) in the area of the respective projection. The projections 13, 18 as well as also possibly provided insulation components may be formed by injection-molding, applying, filling a hardening compound or by gluing or inserting an insulation platelet/pad/pin preferably into a recess in the respective electrode, as will be described in greater detail hereinafter.

In the proximal end portion of the instrument branches 2, 3 proximal spacers are configured in the form of material tongues 21 of a spacer module 19 which has already been described with reference to FIGS. 3 to 6 the description of which shall be referred to.

In the medial portion three evenly spaced insulation components 23, 24, 25 are arranged on the electrode surface 14. On the opposite electrode surface 16 three evenly spaced electrically conductive projections 26, 27, 28 are formed at respective positions. In the medial portion of the electrode surface 15 there are equally formed three evenly space electrically conductive projections 29, 30, 31, while on the opposite electrode surface 17 at respective positions three evenly spaced insulation components 32, 33, 34 are formed. As can be inferred especially from FIG. 8, when the instrument branches 2, 3 are closed, the insulation component 23 abuts on the projection 26, the insulation component 24 abuts on the projection 27 and the insulation component 25 abuts on the projection 28. Correspondingly, the insulation components and projections of the electrode surfaces 15 and 17 get into mutual contact.

FIG. 13 shows an electrosurgical instrument 102 according to another embodiment which differs from the afore-described instrument 1 by the type of instrument, especially the design of the actuating means and the handle. While all of the afore-described different embodiments have been described with reference to the laparoscopic electrosurgical instrument 1 shown in FIG. 1 having a thin elongate shaft 4 and instrument branches 2 and 3 pivotally hinged thereto as a distal jaw part, the afore-described variants of spacer arrangements can be equally realized in connection with the instrument 102 in which, however, two instrument branches 104 and 106 are connected via a slide element 108 and appropriate mimics (not shown in detail). The handle or grip part includes sort of a receiving hole from which the instrument branches are axially and distally projecting. Said hole is dimensioned so that the instrument branches, when they retract via the slide element 108 into the hole, are compressed by the same. When the slide element 108 is advanced in the direction of the hole opening, the instrument branches move out of the hole and open preferably automatically due to a spring bias, for example.

The shape and the size of the spacers may be selected at will as long as all spacers are adapted to each other so that there is always given the same distance between the electrode surfaces at all positions. The spacer may also take a pyramidal (truncated), cylindrical or cube shape. The spacer module after all can be split into plural juxtaposed individual modules each having one material tongue only.

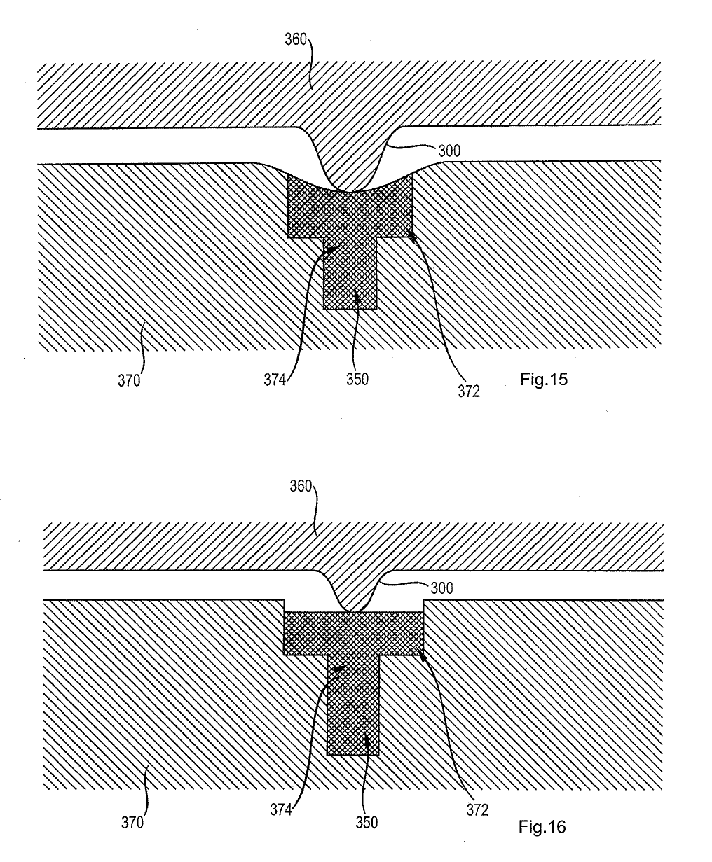

Hereinafter a spacer 300 is described as an example of one of the projections 26, 27, 28, 29, 30, 31 and an insulation component 350 is described in detail as an example of one of the insulation components 23, 24, 25, 32, 33, 34 by way of FIGS. 14 to 16, as they are basically used in afore-described embodiments of a jaw part preferably on the electrode.

As stated already, the spacers or projections 300 according to the present invention made of an electrically conductive material (i.e. electrically conducting) are preferably integrally formed with/connected to an exemplified pertinent electrode 360. The respective projection 300 can be manufactured by appropriate punching and bending or by (punctual) embossing/pressing of the electrode 360 itself. On principle it is also possible, however, to weld, solder or integrally form the electrically conductive projection 300 for example in the form of a cone according to FIGS. 14 to 16 or of a hemi-sphere onto the electrode 360. In this way, high strength between the projection 300 and the electrode 360 as well as a rigid projection are produced at any rate so that the projection formed in this way is largely prevented from being inadvertently scratched or broken off.

On an opposite electrode 370 (of the respective other branch) plate- or disk-shaped recesses or indentations 372 are formed in the area of the projection 300. Said indentations 372 in addition include a central blind hole or through bore 374 in the electrode 370 which extends in the thickness direction of the electrode 370. In this way a kind of mushroom-shaped recess forms in the respective electrode 370 in the longitudinal section according to FIGS. 14 to 16.

A pin/pad or plug is inserted into said recess as an insulation component 350 made of electrically non-conductive material the shape of which is substantially exactly adapted to the recess and which defines the insulation component. As an alternative to this, it is also possible to inject a cast compound of electrically non-conductive material into the recess, which cast compound then hardens.

According to FIG. 14, the plug 350 closes off substantially over the whole surface and flush with the surface of the respective electrode 370. In this way, the plug 350 does not form an external point of contact so that it can be withdrawn from the recess, where necessary. Furthermore, the outer surface of the plug 350 is substantially adjusted to the opposite projection 300 and is sized sufficiently large that the projection 300 safely rests on the plug 350, when the two branches are compressed, without directly contacting the respective electrode.

Each of FIGS. 15 and 16 shows alternative configurations for the plug 350 (insulation component) where according to FIG. 15 a concave outer surface is provided on the plug 350 for causing better centering of the opposite projection 300 upon compressing the branches or where according to FIG. 16 the plug 350 is (slightly) recessed vis-à-vis the surface of the respective electrode so as to absolutely avoid a protrusion.

Finally, it is referred to the fact that the insulation component basically may also take a shape other than the illustrated plug 350. It is possible to design the insulation component to be exclusively flat, i.e. plate-shaped. There is also the option to design the plug 350 in pyramidal or conical shape. Ceramic or plastic material offers itself as a material for the insulation component. Also, between the insulation component (especially the plug 350) and the electrode 370 an intermediate layer may be provided which compensates for different material expansions due to heat between the electrode 370 and the insulation component and in this way prevents the insulation component from breaking or bulging out of the recess 372.

1. An electrosurgical instrument having a jaw part composed of instrument branches which are movable towards each other and on each mutually facing side of which at least one electrode surface is arranged/formed, wherein the movement of the instrument branches relative to each other is limited by a proximal spacer acting on proximal end portions of the instrument branches, a distal spacer acting on distal end portions of the instrument branches and at least one medial spacer acting on a medial portion of the instrument branches, wherein the at least one medial spacer is formed in that at an electrode, a stop elevating from the at least one electrode surface thereof is made of electrically conductive material and is connected electrically conductively to the electrode and interacts with an electrically insulating insulation component on the electrode surface of the opposite electrode; and
wherein the proximal spacer and the distal spacer are made of electrically non-conductive material.

2. The electrosurgical instrument according to claim 1, wherein the instrument includes exactly one medial spacer.

3. The electrosurgical instrument according to claim 1, wherein the insulation component includes a contact face for the stop and a mushroom-shaped body, wherein the surface width of the contact face in the electrode plane protrudes from all sides of the stop so that the stop gets no electric contact with the electrode including the insulation component.

4. The electrosurgical instrument according to claim 1, wherein the electrode including the insulation component has at least one recess on its surface into which the insulation component is inserted or which is molded with an electrically insulating material which after solidifying forms the insulation component.

5. The electrosurgical instrument according to claim 1, wherein the insulation component is flush with the electrode surface or has a concavity or is recessed with respect to the electrode surface.

6. The electrosurgical instrument according to claim 1, wherein at least one of the proximal spacer and the distal spacer is arranged exclusively outside an area provided for the treatment of the tissue.

7. The electrosurgical instrument according to claim 1, further comprising at least one of the following:
the proximal spacer is formed by a projection arranged at a proximal end portion of an instrument branch between two electrode surfaces of said instrument branch and facing the other instrument branch; and
the distal spacer is formed by a projection arranged at a distal end portion of an instrument branch between two electrode surfaces of said instrument branch and facing the other instrument branch.

8. The electrosurgical instrument according to claim 1, wherein
at least one of the distal spacer and the proximal spacer is formed by one projection provided on an electrode surface or by plural projections provided on respective different electrode surfaces.

9. The electrosurgical instrument according to claim 1, wherein
the proximal spacer is formed by a spacer module formed separately from the instrument branches and having at least one electrically non-conductive material tongue which is clamped between the instrument branches in a closing position.

10. The electrosurgical instrument according to claim 9, wherein
the spacer module is rotatably supported in a pivot joint of a pivoting instrument branch and accordingly is encompassed by wall-shaped joint portions of the pivoting instrument branch which define a receiving cavity for the spacer module.

11. The electrosurgical instrument claim 1, wherein
the height of the proximal spacer, of the distal spacer and of the medial spacer corresponds to a predetermined minimum distance between the instrument branches in the closed position.

12. The electrosurgical instrument according to claim 1, wherein
the spacers are spaced equally from each other.

13. The electrosurgical instrument according to claim 1, wherein
the spacers are formed on each electrode surface.
